SEQUENCE LISTING (7 sequences)

SEQ ID NO: 1 - DNA, 786 bp, Lactococcus lactis
atgaaaacacttatcactggcgaaaataaaggaattggttttgcacttgctcaaaaccttggtcatcgcggctatgaagtcttggtcggagcccgtaatgaaacgcgtggacaagaagccgttgaaaagctgaaagccgaaggaattactgccaaatttgtcaaagtagatttagatgacttaaatcaattgacgagtctgtcagcactaacagatattgatttacttattaataatgctggaatttctgggaatattcattcagataaagggcaccttgatatggaaaaatcagcttttgattattcaacaactgatttagaagaaacaattaaaaccaattttttgggaacacatgctgtgatcaaagaacttcttccacattcactgacagaaaatgctaaaattattaacatcaccgtacccgtttctcaagaatattggatgccacttgcttatgtcacttcaaaagctgcacaaaatgccatgacctttgcttttggtcatcaattcaaaaaagataaatctaaaaaacaaatctttgctgttatgcctggtgcagttgcaacagatttgaacggtgcaaaagttggggatagtccgtttgtaaaatctcctgaagaaacagctcaaatgattagtaaatttatttttgatgacaaaaatcataatgctcaaattattaattttgacggaacaatttatgataactatgaaccaggcttacgtaacaaagtcattaaagatgtcgctaaaaatggtttaaagcgtaaatttaaaaaataa

SEQ ID NO: 2 - protein, 261 aa, Lactococcus lactis
MKTLITGENKGIGFALAQNLGHRGYEVLVGARNETRGQEAVEKLKAEGITAKFVKVDLDDLNQLTSLSALTDIDLLINNAGISGNIHSDKGHLDMEKSAFDYSTTDLEETIKTNFLGTHAVIKELLPHSLTENAKIINITVPVSQEYWMPLAYVTSKAAQNAMTFAFGHQFKKDKSKKQIFAVMPGAVATDLNGAKVGDSPFVKSPEETAQMISKFIFDDKNHNAQIINFDGTIYDNYEPGLRNKVIKDVAKNGLKRKFKK

SEQ ID NO: 3 - protein, 268 aa, Lactococcus brevis
MSNRLDGKVAIITGGTLGIGLAIATKFVEEGAKVMITGRHSDVGEKFVEEGAKVMITGRHSDVGEKAAKSVGTPDIFFHDSSDEDGWTKLFDATEKAFGPVSTLVNNAGIAVNKSVEETTTAEWRKLLAVNLDGVFFGTRLGIRMKNKGLGASIINMSSIEGFVGDPSLGAYNASKGAVARIMSKSAALDCALKDYDVRVNTVHPGYIKTPLVDDLPGAEEAMSRTKTPMGHIGEPNDIAYICVYLASNESKFATGSEFVVDGGYTAQ

SEQ ID NO: 4 - protein, 259 aa, Lactococcus lactis
MKTLITGENKGIGFALAQNLGHRGYEYLVGARNETRGQEAVEKLKAEGITAKFVKVDLDDLNQLTSLALTDIDLINNAGISGNIHSDKGHLDMEKSAFDYSTTDLEETIKTNFLGTHSVIKELLPHSLTENAKIINITVPVSQEYWMPLAYVTSKAAQNAMTFAFGHQFKKDKSKKQIFAVMPGAVATDLNGAKVGDSPFVKSPEETAQMISKFIFDDKNHNAQIINFDGTIYDNYEPGLRNKVIKDVAKNGLKRKFKK

SEQ ID NO: 5 - DNA, 37 bp, artificial
  primer
gcgcgaattcatgaaaacacttatcactggcgaaaat

SEQ ID NO: 6 - DNA, 37 bp, artificial
  primer
gcgcaagcttttattttttaaatttacgctttaaacc

SEQ ID NO: 7 - DNA, 47 bp, artificial
  primer
gcaagcttctagtggtggtggtgttttttaaatttacgctttaaacc

(19) United States
(12) Patent Application Publication
(10) Pub. No.: US 2006/0240535 A1
(43) Pub. Date: Oct. 26, 2006

(54) Carbonyl reductase from lactococcus lactis

(75) Inventors: Andreas Sebastian Bommarius, Atlanta, GA (US); Bettina Renate Else Bommarius, Atlanta, GA (US)

Correspondence Address: PATREA L. PABST;PABST PATENT GROUP LLP, 400 COLONY SQUARE, SUITE 1200, ATLANTA, GA, 30361, US

(73) Assignee: GEORGIA TECH RESEARCH CORPORATION, Atlanta, GA (US)

(21) Appl. No.: 11/271,585

(22) Filed: Nov. 10, 2005

Related U.S. Application Data
(60) Provisional application No. 60/626,654, filed on Nov. 10, 2004.

(51) Int. Cl. C12P 7/22; C12P 7/02; C12P 21/06; C12P 19/30; C07H 21/04; C12N 9/02

(52) U.S. Cl. 435/155; 435/156; 435/189; 435/69.1; 435/320.1; 435/325; 530/388.26; 536/23.2; 435/89

(57) ABSTRACT

Compositions and methods of using a carbonyl reductase from Lactococcus lactis (LLCR or LL-CR) are provided. One aspect provides an isolated polypeptide from Lactococcus lactis, wherein the polypeptide is an (R)-specific carbonyl reductase or a fragment thereof and reduces acetophenone or alph,beta-unsaturated carbonyl compound to form a reduced R enantiomeric product. In certain aspects, LLCR is stable in from about 1.0% to about 60% organic solvent or more than 10%, 20%, 30%, 40% or 50% organic solvent.

### CROSS-REFERENCE TO RELATED APPLICATION

This application claims benefit of and priority to U.S. Provisional Patent Application No. 60/626,654 filed on Nov. 10, 2004, which is incorporated by reference in its entirety.

### BACKGROUND

1. Technical Field

This application is generally directed to carbonyl reductase from Lactococcus lactis, compositions containing the carbonyl reductase or fragments thereof, and methods of use thereof.

2. Background

The synthesis of specific optical isomers of therapeutic compounds is important to the pharmaceutical, life science, and chemical industries. Regulations enacted by the U.S. Food and Drug Administration require characterization of each enantiomer of a pharmaceutical compound. Additionally, the FDA requires 99% optical purity of approved enantiomeric drugs. To obtain characterization data of each enantiomer, methods for easily producing specific enantiomers of pharmaceutical compounds or optical intermediates in large quantities are needed. One method for producing specific enantiomers of compounds includes the use of enzymes such as carbonyl reductase. In the chemical industry, enantiomerically pure amino acids, aminoalcohols, amines, alcohols, and epoxides are produced using enzymatic and fermentation processes.

Most known carbonyl reductases produce (S)-specific products and do not function well in organic solvents. The few known (R)-specific reductases likewise have poor activity in organic solvents. Because the pharmaceutical industry uses organic solvents extensively in the production of pharmaceutical compounds, the currently known carbonyl reductases are of limited use in this industry. Moreover, the discovery of new (R)-specific reductases has been difficult because there is no discernible motif for (R)-specificity shared between the known (R)-specific reductases.

### SUMMARY

Compositions and methods of using a carbonyl reductase from Lactococcus lactis (LLCR or LL-CR) are provided. One aspect provides an isolated polypeptide from Lactococcus lactis, wherein the polypeptide is an (R)-specific carbonyl reductase or a fragment thereof and reduces acetophenone or alph,beta-unsaturated carbonyl compound to form a reduced R enantiomeric product. In certain aspects, LLCR is stable in from about 1.0% to about 60% organic solvent or more than 10%, 20%, 30%, 40% or 50% organic solvent.

Another aspect provides an isolated and purified microbial polypeptide that has at least 80% sequence identity to a polypeptide having an amino acid sequence of SEQ ID NO:2, wherein the polypeptide has R carbonyl reductase activity and forms a tetramer.

Still another aspect provides a method for reducing an acetophenone or an alph,beta-unsaturated carbonyl compound by reacting the acetophenone or alpha, beta-unsaturated carbonyl compound with an isolated polypeptide comprising a sequence having at least about 80% sequence identity to SEQ ID NO:2 or fragment thereof in combination with NADP(H) to form a reduced R enantiomeric product.

Other aspects of the disclosed subject matter will become apparent in view of the appended Figures and Detailed Description. The Figures are provided to further explain the disclosed subject matter and are not intended to limit the invention.

### BRIEF DESCRIPTION OF THE FIGURES

FIG. 1 shows a protein sequence comparison between an exemplary carbonyl reductase from L. lactis, an (R)— specific carbonyl reductase from L. brevis, and a putative carbonyl reductase from L. lactis.

FIG. 2 shows a line graph indicating the level of enzymatic activity of an exemplary carbonyl reductase for acetophenone according the present disclosure in solutions containing various organic solvents at increasing concentrations.

FIG. 3 shows a line graph indicating the level of enzymatic activity of an exemplary carbonyl reductase for ethyl 3-oxohexanoate in solutions containing various organic solvents at increasing concentrations.

FIG. 4 shows a line graph comparing the activity of an exemplary carbonyl reductase from L. lactis to a horse liver ADH.

FIG. 5 shows a line graph indicating the pH optima of an exemplary carbonyl reductase from L. lactis.

FIG. 6 shows a line graph indicating the pH stability of an exemplary carbonyl reductase from L. lactis.

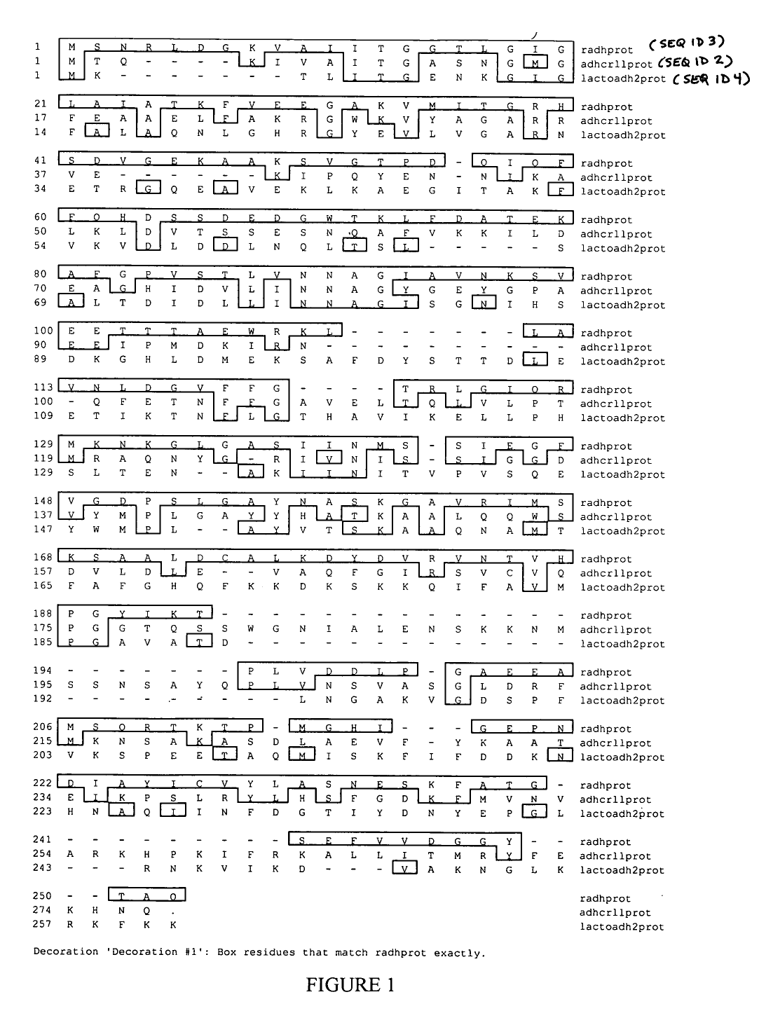

### DETAILED DESCRIPTION

### DEFINITIONS

In describing and claiming the disclosed subject matter, the following terminology will be used in accordance with the definitions set forth below.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48:1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “purified” and like terms relate to the isolation of a molecule or compound in a form that is substantially free (at least 60% free, preferably 75% free, and most preferably 90% free) from other components normally associated with the molecule or compound in a native environment.

“Operably linked” refers to a juxtaposition wherein the components are configured so as to perform their usual function. For example, control sequences or promoters operably linked to a coding sequence are capable of effecting the expression of the coding sequence.

As used herein, the term “vector” is used in reference to a vehicle used to introduce a nucleic acid sequence into a cell. A viral vector is virus that has been modified to allow recombinant DNA sequences to be introduced into host cells or cell organelles.

As used herein, the term “polynucleotide” generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

### Embodiments

Embodiments of the present disclosure are generally directed to a carbonyl reductase from Lactococcus lactis, methods of its use, and compositions containing the enzyme or a fragment thereof. The disclosed carbonyl reductase, variants, or fragments thereof have industrial applications, for example pharmaceutical applications in biocatalysis to generate chiral building blocks for pharmaceutical drugs. In pharmaceutical use preferences for certain enzymatic catalysts over chemical variants exist, partly because of increasing environmental restrictions, but also because more and more enzymes have proven to provide better results.

In one embodiment, the disclosed carbonyl reductase from Lactococcus lactis is (R)— specific, whereas most of the known carbonyl reductases in use are (S)-specific. The disclosed enzyme displays a broad substrate specificity which makes it a versatile catalyst, but also includes aromatic side chains, a feature often needed in producing pharmaceutical drugs requiring organic solvents. Exemplary substrates include, but are not limited to aliphatic ketones, aromatic ketones, alpha ketoesters, beta ketoesters, or a combination thereof as well as secondary alcohols. A list of representative substrates is provided in Table 1. This enzyme is also useful in organic solvents to convert substrates having limited solubility in water, for example hydrophobic compounds containing a carbonyl group.

The purified LL-CR is an annotated protein with just under 30 kDa per subunit by SDS-PAGE (28.8 kDa as calculated from verified sequence) and requires NADP(H) as cofactor. The enzyme reduces its substrate to produce a compound having an (R)-specific alcohol functional group. Alternatively, the enzyme can oxidize (R)-specific secondary alcohols, such as (R)-2-hexanol and (R)-1-phenyl-1-ethanol, but not the respective (S)-secondary alcohols to the respective ketones, such as 2-hexanone and acetophenone, and thus, the enzyme is (R)-specific. The LL-CR has pH and T optima of 6.5 and 40° C. and measured K_{M} values of 0.5 mM for acetophenone and 0.03 mM for NADPH and vₘₐₓ=80 U/mg pure protein at pH 7.0 and 30° C. and a calculated k_{cat} of 24 s⁻¹.

One embodiment provides a polypeptide encoded by a nucleic acid having at least about 80%, 85%, 90%, 95%, 99% or more sequence identity to the nucleic acid:

| | |
|---|---|
| atgaaaacacttatcactggcgaaaataaaggaattg | (SEQ ID NO:1) |
| gttttgcacttgctcaaaaccttggtcatcgcggcta | |
| tgaagtcttggtcggagcccgtaatgaaacgcgtgga | |
| caagaagccgttgaaaagctgaaagccgaaggaatta | |
| ctgccaaatttgtcaaagtagatttagatgacttaaa | |
| tcaattgacgagtctgtcagcactaacagatattgat | |
| ttacttattaataatgctggaatttctgggaatattc | |
| attcagataaagggcaccttgatatggaaaaatcagc | |
| ttttgattattcaacaactgatttagaagaaacaatt | |
| aaaaccaattttttgggaacacatgctgtgatcaaag | |
| aacttcttccacattcactgacagaaaatgctaaaat | |
| tattaacatcaccgtacccgtttctcaagaatattgg | |
| atgccacttgcttatgtcacttcaaaagctgcacaaa | |
| atgccatgacctttgcttttggtcatcaattcaaaaa | |
| agataaatctaaaaaacaaatctttgctgttatgcct | |
| ggtgcagttgcaacagatttgaacggtgcaaaagttg | |
| gggatagtccgtttgtaaaatctcctgaagaaacagc | |
| tcaaatgattagtaaatttatttttgatgacaaaaat | |
| cataatgctcaaattattaattttgacggaacaattt | |
| atgataactatgaaccaggcttacgtaacaaagtcat | |
| taaagatgtcgctaaaaatggtttaaagcgtaaattt | |
| aaaaaataa. | |

Another embodiment provides a polypeptide comprising an amino acid sequence having at least about 80%, 85%, 90%, 95%, 99% or more sequence identity to: MKTLITGENKGIGFALAQNLGHRGYEVLVGARNETRGQEAVEKLKAEGITAKFVKVDL DDLNQLTSLSALTDIDLLINNAGISGNIHSDKGHLDMEKSAFDYSTTDLEETIKTNFLGTH AVIKELLPHSLTENAKIINITVPVSQEYWMPLAYVTSKAAQNAMTFAFGHQFKKDKSKK QIFAVMPGAVATDLNGAKVGDSPFVKSPEETAQMISKFIFDDKNHNAQIINFDGTIYDNY EPGLRNKVIKDVAKNGLKRKFKK (SEQ ID NO:2) or a fragment thereof.

Still another embodiment provides an isolated and purified a microbial polypeptide that has at least 95% sequence identity to a polypeptide having an amino acid sequence of SEQ ID NO:2, wherein the polypeptide has (R)— carbonyl reductase activity. The polypeptide reduces aliphatic ketones, aromatic ketones, alpha ketoesters, beta ketoesters, or a combination thereof, but does not reduce keto acids or act on primary alcohols. In one embodiment, LLCR forms a tetramer.

The disclosed carbonyl reductase generates (R)— specific alcohol functional groups by reducing the carbonyl group on aliphatic ketones, aromatic ketones, alpha ketoesters, beta ketoesters. The resulting enantiomeric product can be used as an intermediate in the synthesis of a specific enantiomeric compound, for example an enantiomeric pharmaceutical compound. The enantiomeric product can be further concentrated or purified as needed using methods known in the art. In one embodiment, about 50%, 60%, 70%, 80%, 85%, 90%, 95%, 99% or more of the aliphatic ketones, aromatic ketones, alpha ketoesters, beta ketoesters in a reaction are reduced to produce an enantiomeric product.

In one embodiment, the carbonyl reductase is stable in a solution comprising an organic solvent. Stable refers to retaining reductase activity in the presence of organic solvents. Representative organic solvents include, but are not limited to acetonitrile, HEPES, triethanolamine, dioxane, and tetrahydrofuran. In certain embodiments, LLCR is stable in from about 1.0% to about 60% organic solvent or more than 10%, 20%, 30%, 40% or 50% organic solvent.

One embodiment provides an LLCR that retains carbonyl reductase activity in solutions comprising about 60% organic solvent compared to carbonyl reductase in the absence of organic solvents. Another embodiment provides an LLCR that retains at least 30% reductase activity in the presence of about 15% organic solvent, or at least 15% activity in about 30% organic solvent, or at least about 5% activity in 40% organic solvent.

Another embodiment provides a tetramer comprising subunits comprising the polypeptide having at least 95% sequence identity to SEQ ID NO:2.

Still another embodiment provides a vector comprising a nucleic acid of SEQ ID NO:1 operably linked to a promoter. The vector expresses the encoded polypeptide in a form that retains carbonyl reductase activity in the presence of about 1% to about 60% organic solvent.

Methods of Use

The LLCR or a fragment thereof can be used to generate (R)— specific alcohol functional groups on carbonyl compounds by reducing at least one carbonyl of the compound. One embodiment provides a method for reducing a hydrophobic ketone by reacting or contacting the ketone with LLCR or a fragment thereof in combination with NADP(H) in a solution comprising about 1% to about 60%, typically about 10% to about 60%, even more typically about 15% to about 60% of an organic solvent. Generally, the solution is a mono-phasic aqueous-organic solution. Once the compound having an (R)-specific alcohol functional group is produced, it can be isolated, concentrated, or reacted with at least a second reagent to produce a desired enantiomeric product. In certain embodiments, the LLCR retains at least about 40% carbonyl reductase activity compared to activity without the presence of the organic solvent, or about 20% carbonyl reductase activity in the presence of about 30% organic solvent, or retains reductase activity in the presence of about 40% to about 60% organic solvent.

Another embodiment provides a method for reducing an acetophenone or an alph,beta-unsaturated carbonyl compound by reacting the acetophenone or alpha, beta-unsaturated carbonyl compound with an isolated polypeptide comprising a sequence having at least about 80% sequence identity to SEQ ID NO:2 or fragment thereof in combination with NADP(H) to form a reduced (R)-enantiomeric product. Generally, the reaction is carried on in a buffered solution optionally comprising an organic solvent, for example from about 1% to about 60% organic solvent. NADP(H) is added as a cofactor, and the polypeptide reduces the substrate acetophenone or alpha, beta-unsaturated carbonyl compound. The reduced substrate comprises an (R)— alcohol functional group and is therefore an enantiomeric product.

Another embodiment provides a method for producing NADPH by contacting a secondary alcohol with LLCR or a fragment thereof in the presence of NADP+ optionally in the presence of from about 1% to about 60% organic solvent, about 10% to about 60% organic solvent, or about 15% to about 60% organic solvent. A representative secondary alcohol is isopropanol.

Another embodiment provides a composition and method for attaching the LLCR on a solid support through adsorption, electrostatic interactions, covalent bonding, or entrapment. Such a solid support can be employed as a single bead or in a packed-bed reactor.

Another embodiment provides a composition and method for using LLCR in a gel, such as those created with aqueous buffer, hydrocarbon, and surfactant molecules, such as alkyl-ethoxyalcohols of structure (CₙH₂ₙ₊₁)ₘ(EO)ₚ—OH, where m, n, and p each are greater than or equal to 2. Such gels are useful for preserving activity and stability, allowing use in liquid media, and in uses requiring applications of the enzyme-gel system in a layer, such as in coating or personal care applications.

Another embodiment provides a method for using LLCR as a biosensor for NADPH— specific events in unknown probes, such as aqueous solutions, tissues, or fermentation samples.

Modifications of LLCR

Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

In one embodiment, LLCR can be modified to increase its stability in organic solvents by making conservative substations with amino acids having non-polar side chains.

Expression and Purification of Encoded Proteins

The cDNA species specified in SEQ ID NO:1 can be expressed as encoded peptides or proteins. The engineering of DNA segment(s) for expression in a prokaryotic or eukaryotic system may be performed by techniques generally known to those of skill in recombinant expression. It is believed that virtually any expression system may be employed in the expression of the claimed nucleic acid sequences.

Both cDNA and genomic sequences are suitable for eukaryotic expression, as the host cell will generally process the genomic transcripts to yield functional mRNA for translation into protein. Generally speaking, it may be more convenient to employ as the recombinant gene a cDNA version of the gene. It is believed that the use of a cDNA version will provide advantages in that the size of the gene will generally be much smaller and more readily employed to transfect the targeted cell than will a genomic gene, which will typically be up to an order of magnitude larger than the cDNA gene. However, the inventor does not exclude the possibility of employing a genomic version of a particular gene where desired.

As used herein, the terms “engineered” and “recombinant” cells are intended to refer to a cell into which an exogenous DNA segment or gene, such as a cDNA or gene has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic DNA, and also include genes positioned adjacent to a promoter not naturally associated with the particular introduced gene.

To express a recombinant encoded protein or peptide, whether mutant or wild-type, in accordance with the present invention one would prepare an expression vector that comprises one of the claimed isolated nucleic acids under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors.

Certain examples of prokaryotic hosts are E. coli strain RR1, E. coli LE392, E. coli B, E. coli 1776 (ATCC No. 31537) as well as E. coli W3110 (F—, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using pBR322, a plasmid derived from an E. coli species. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector that can be used to transform host cells, such as E. coli LE392.

Further useful vectors include pIN vectors and pGEX vectors, for use in generating glutathione S-transferase (GST) soluble fusion proteins for later purification and separation or cleavage. Other suitable fusion proteins are those with β-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autograph californica nuclear polyhidrosis virus (AcNPV) is used as a vector to express foreign genes. The virus grows in Spodoptera frugiperda cells. The isolated nucleic acid coding sequences are cloned into non-essential regions (for example the polyhedron gene) of the virus and placed under control of an AcNPV promoter (for example, the polyhedron promoter). Successful insertion of the coding sequences results in the inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, NIH3T3, RIN and MDCK cell lines. In addition, a host cell may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cell lines or host systems can be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HinDIII site toward the BglI site located in the viral origin of replication.

In cases where an adenovirus is used as an expression vector, the coding sequences may be ligated to an adenovirus transcription/translation control complex, e.g., the late promoter and. tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited, to the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

It is contemplated that the isolated nucleic acids of the invention may be “overexpressed”, i.e., expressed in increased levels relative to its natural expression in human cells, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

Purification of Expressed Proteins

Further aspects of the present invention concern the purification, and in particular embodiments, the substantial purification, of an encoded protein or peptide. The term “purified protein or peptide ” as used herein, is intended to refer to a composition, isolatable from other components, wherein the protein or peptide is purified to any degree relative to its naturally-obtainable state, i.e., in this case, relative to its purity within a hepatocyte or p-cell extract. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

Preparation of Antibodies Specific for Encoded Proteins

For some embodiments, it will be desired to produce antibodies that bind with high specificity to the protein product(s) of an isolated nucleic acid selected from the group comprising the sequences in SEQ ID NO:1, or any mutant or variant of LLCR. Means for preparing and characterizing antibodies are well known in the art (See, e.g., Antibodies: A Laboratory Manual, Cold Spring Harbor Laboratory, 1988, incorporated herein by reference).

In one embodiment, the antibody is specific for the tetramer of LLCR and optionally, has less than 10% binding activity to an individual subunit compared to the binding activity to the tetramer.

Methods for generating polyclonal antibodies are well known in the art. Briefly, a polyclonal antibody is prepared by immunizing an animal with an antigenic composition and collecting antisera from that immunized animal. A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, may also be given. The process of boosting and titering is repeated until a suitable titer is achieved. When a desired level of immunogenicity is obtained, the immunized animal can be bled and the serum isolated and stored, and/or in some cases the animal can be used to generate monoclonal antibodies (MAbs). For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or the desired antibody fraction may be purified by well-known methods, such as affinity chromatography using another antibody or a peptide bound to a solid matrix.

Monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner that effectively stimulates antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages, but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and have enzyme deficiencies that render them incapable of growing in certain selective media that support the growth of only the desired fused cells (hybridomas).

Any one of a number of myeloma cells may be used, as are known to those of skill in the art (Goding, 1986). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON—HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods is also appropriate.

Fusion procedures usually produce viable hybrids at low frequencies, about 1×10⁻⁶ to 1×10⁻⁸. However, this low frequency does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and thus they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. MAbs produced by either means may be further purified, if desired, using filtration, centrifugation and various chromatographic methods such as HPLC or affinity chromatography.

Large amounts of the monoclonal antibodies of the present invention may also be obtained by multiplying hybridoma cells in vivo. Cell clones are injected into mammals that are histocompatible with the parent cells, e.g., syngeneic mice, to cause growth of antibody-producing tumors. Optionally, the animals are primed with a hydrocarbon, especially oils such as pristane(tetramethylpentadecane) prior to injection.

In accordance with the present invention, fragments of the monoclonal antibody of the invention can be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention can be synthesized using an automated peptide synthesizer, or by expression of full-length gene or of gene fragments in E. coli.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents or by reaction with an isothiocyanate. Conjugates with metal chelates are similarly produced. Other moieties to which antibodies may be conjugated include radionuclides such as ³H, ¹²⁵I, 131I, ³²P, ³⁵S, ¹⁴C, ⁵¹Cr, ³⁶Cl, ⁵⁷Co, ⁵⁸Co, ⁵⁹Fe, ⁷⁵Se, ¹⁵²Eu, and ⁹⁹mTc, are other useful label that can be conjugated to antibodies. Radioactively labeled monoclonal antibodies of the present invention are produced according to well-known methods in the art. For instance, monoclonal antibodies can be iodinated by contact with sodium or potassium iodide and a chemical oxidizing agent such as sodium hypochlorite, or an enzymatic oxidizing agent, such as lactoperoxidase. Monoclonal antibodies according to the invention may be labeled with technetium-⁹⁹ by ligand exchange process, for example, by reducing pertechnate with stannous solution, chelating the reduced technetium onto a Sephadex column and applying the antibody to this column or by direct labelling techniques, e.g., by incubating pertechnate, a reducing agent such as SnCl₂, a buffer solution such as sodium-potassium phthalate solution, and the antibody.

It will be appreciated by those of skill in the art that monoclonal or polyclonal antibodies specific for LLCR or a fragment thereof will have utilities in several types of applications. These can include the purification of the polypeptide as well as detection of the polypeptide in a sample. The skilled practitioner will realize that such uses are within the scope of the present invention.

The following Examples are provided to further describe various embodiments of the disclosed subject matter and are not intended to limit the scope of the invention.

### EXAMPLES

### Example 1

The DNA sequence was retained from the fully sequenced genome of Lactococcus lactis, available at Genebank, Accession number: NC_{—}002662, which is incorporated by reference in its entirety. The search included typical consensus sequences common to short-chain dehydrogenases like GXXGXG (part of the cofactor binding sequence) and LXYXTS (conserved region of the active site).

Primers were generated to the N— and C-terminal parts of the gene and a standard PCR reaction was performed using genomic DNA preparations from the wildtype Lactococcus lactis strain (ATCC 19435D or 29146).

Primer sequences used:

(SEQ ID NO:5)

gcgcgaattcatgaaaacacttatcactggcgaaaat 5′
adhlactos

(SEQ ID NO:6)

gcgcaagcttttattttttaaatttacgcttt
aaacc 3′ adhlactoas

The primers already included appropriate restriction sites for cloning purposes derived from restriction maps created with the DNA sequence of CRLL. The successfully amplified gene of 786 bp was cloned into pBluescript vector SK-(Stratagene) and after sequence verification, subcloned into the expression vector pkk223-3 (Amersham) using the same restriction sites. For further facilitation of purification a C-terminal His-tag was artificially added to the sequence using an additional primer, called 3′ lactoadhhisas and again cloned into the vector pkk233-3.

Prime sequence: gcaagcttctagtggtggtggtgttttttaaatttacgctttaaacc 3′ lactoadhhisas (SEQ ID NO:7). CRLL DNA amplification resulted in a band between 500 and 1000 bp matching the proposed molecular weight of the CRLL-gene.

### Example 2

The construct crll-pkk223-3 as well as crll-his-pkk223-3 were both transformed separately into the E. coli host strain HB101 for expression. Tested clones were positive for CRLL activity and the plasmids as well as the bacterial strains were stocked. Initial CRLL activity was measured using 10 mM Acetophenone in 50 mM TEA pH 7.5 and 0.2 mM NADPH at 30° C. Activity is defined as decrease of NADPH absorption proportional to enzyme activity followed the Lambert-Beer Law.

1 L shaker flasks were used for 200 mL cultures, incubated at 30 C and shaken with 200 rpm. Induction of enzyme expression was performed after reaching OD 1 with 1 mM IPTG and expression occurred for an additional 4 h incubation under the same conditions. Cells were harvested using a Beckman centrifuge and the cell pellets stored until further use.

The cell pellets were lysed using ultrasonification and the following lysis buffer: 100 mM TEA pH 7.5 with 1 mM EDTA. The cell suspension was subjected to ultrasonication for 10 min with constant cooling at 4 C. The lysed cells were spun at 13000 rpm for 15 min to remove cell debris and obtain the crude extract harboring the soluble expressed CRLL. Since both the CRLL and the CRLL-his proteins behaved identically, purification was only performed on the his-tagged version of CRLL.

### Example 3

500 ul of the crude extract from Example 2 was mixed with 2 ml of Ni—NTA agarose (Qiagen) as a batch and incubated at 4 C for 2 h under rotation. This ensured affinity binding of the his-tagged protein to the Ni ligand. The agarose was poured into a small column and the column was subsequently washed with increasing stringency.

Washing steps:

| | |
|---|---|
| 1. wash | 10 column volumes of 50 mM TEA pH 7.5 + 150 mM NaCl |
| 2. wash | 10 column volumes of 50 mM TEA pH 7.5 + 1 mM imidazole |
| 3. wash | 10 column volumes of 50 mM TEA pH 7.5 + 2 mM imidazole |

The CRLL-his protein was eluted from the column in 1 ml fractions using 50 mM TEA pH 7.5+10 mM imidazole.

his-tagged protein (0.5 mL, 42.7 mg/mL) charged onto Ni—NTA column (2 mL);

eluted during wash: 15.6 U/mg protein=16.6%; elution with 10 mM imidazole: 118 U/mg, 74% recovery.

Table 1 shows various substrates for the isolated carbonyl reductase and the respective activity for that substrate.

| | altern. | Activity | Protein | spec. Act. |
|---|---|---|---|---|
| Substrate (20 mM) | name | (U/mL) | (mg/mL) | (U/mg) |
| hydroxyacetone | | 0.77 | 0.37 | 2.08108108 |
| acetone | | 8.36 | 0.37 | 22.5945946 |
| 3-methyl-2-butanone | MIPK | 12.06 | 0.37 | 32.5945946 |
| pinacolone | | 22.5 | 6.6 | 3.40909091 |
| 2-hexanone | | 1.28 | 0.37 | 3.45945946 |
| 4-methyl-2-pentanone | MIBK | 21.7 | 6.6 | 3.28787879 |
| 2,4-pentane-dione | | 90.77 | 6.6 | 13.7530303 |
| acetonylacetone | acac | 57.88 | 6.6 | 8.76969697 |
| cyclohexanone | | 49 | 6.6 | 7.42424242 |
| 3,3,5-trimethyl-CHone | | 0.24 | 0.37 | 0.64864865 |
| t-butylcyclohexanone | | 0 | 0.37 | 0 |

| Substrate (20 mM) | altern. | Activity | Protein | spec. Act. |
|---|---|---|---|---|
| acetophenone | | 53 | 6.6 | 8.03030303 |
| propiophenone | | 2.2 | 5.5 | 0.4 |
| tetralone | | 3.2 | 6.6 | 0.48484848 |
| 1-indanone | | 0 | 0.37 | 0 |
| 1-decalone | | 5.29 | 0.37 | 14.2972973 |
| Phenoxy-2-propanone | | 10.61 | 0.37 | 28.6756757 |
| ketopantolactone | | 9.49 | 0.37 | 25.6486486 |
| pyruvate | | 1.8 | 0.37 | 4.86486486 |
| ethyl pyruvate | | 16.32 | 6.6 | 2.47272727 |
| benzoylformate | | 0 | 0.37 | 0 |
| Methyl benzoylformate | | 3.86 | 6.6 | 0.58484848 |
| ethyl benzoylformate | | 11.25 | 6.6 | 1.70454545 |
| phenylpyruvate | | 0 | 6.6 | 0 |
| Methyl acetoacetate | | 36.7 | 6.6 | 5.56060606 |
| ethyl acetoacetate | | 7.07 | 0.37 | 19.1081081 |
| ethyl 3-oxohexanoate | | 9.64 | 0.37 | 26.0540541 |
| ethyl benzoylacetate | | 4.98 | 0.37 | 13.4594595 |
| propionaldehyde | | 69 | 6.6 | 10.4545455 |
| butyraldehyde | | 12.7 | 0.37 | 34.3243243 |
| hexylaldehyde | | 45.82 | 6.6 | 6.94242424 |
| octylaldehyde | | 10.93 | 6.6 | 1.65606061 |
| benzaldehyde | | 8.3 | 6.6 | 1.25757576 |
| 1,2-propanediol | | 0.025 | 0.37 | 0.06756757 |
| ethoxyethanol | | 0.0128 | 0.37 | 0.03459459 |
| diethyleneglycol | | 0.38 | 0.37 | 1.02702703 |

### Example 4

As LL-CR requires organic buffers for stability, such as HEPES or triethanolamine, runs were conducted in partially organic systems using a buffer system of 200 mM NH₄CHO₃ and 50 mM HEPES at pH 7.0.

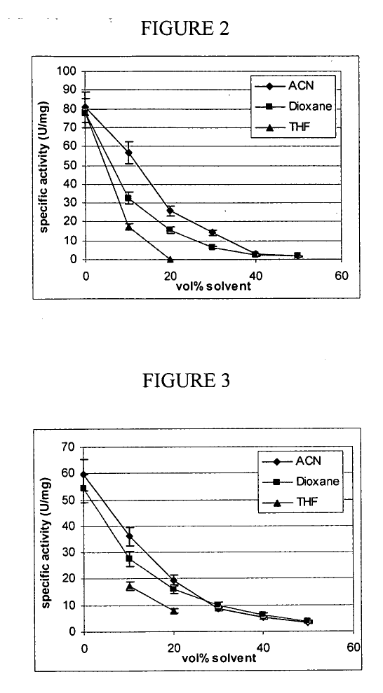

LL-CR reduces a wide range of aliphatic and aromatic ketones, α- and β-keto esters but not keto acids. Acetophenone and ethyl 3-oxohexanoate were studied in detail as model substrates, which are still sufficiently water-soluble to achieve saturation but already preferentially soluble in buffer-organic mixtures. We found solubility to increase exponentially with acetonitrile (ACN) content x_{ACN} for acetophenone {y=0.016·exp(0.0419·x_{ACN})}as well as for ethyl 3-oxo hexanoate {y=0.0117·exp(0.0534·x_{ACN})}. Specific activities of acetophenone and ethyl 3-oxohexanoate as a function of solvent content are shown in FIGS. 2 and 3 respectively.

Purified CRLL activity was measured in aqueous-organic tunable solvents (OATS) using 10 mM acetophenone (FIG. 2) or 10 mM ethyl 3-oxohexanoate (FIG. 3) in 200 mM NH₄HCO₃/50 mM HEPES, pH 7.0, 0.25 mM NADPH and 0.2 mM NADPH at 30° C. (FIG. 2). Activity is defined as decrease of NADPH absorption proportional to enzyme activity followed the Lambert-Beer Law.

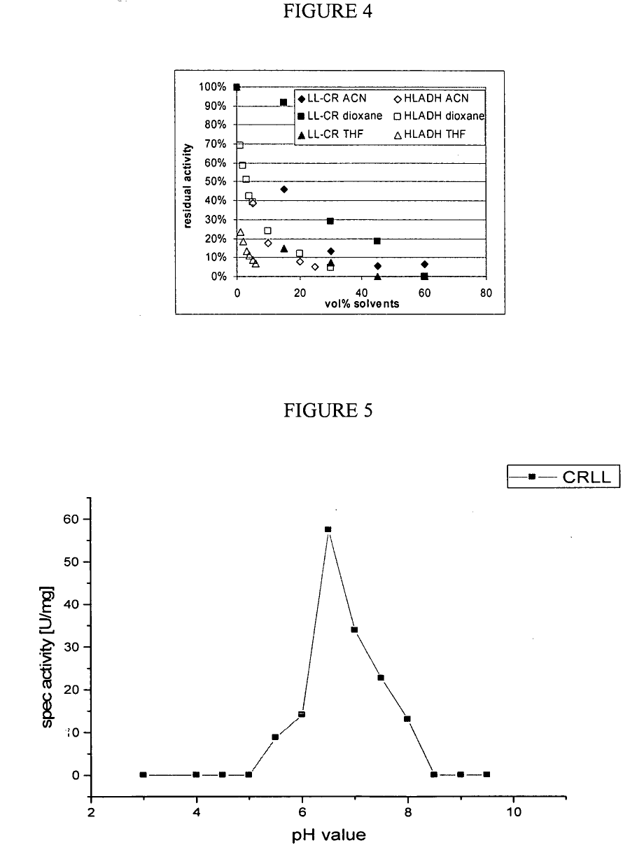

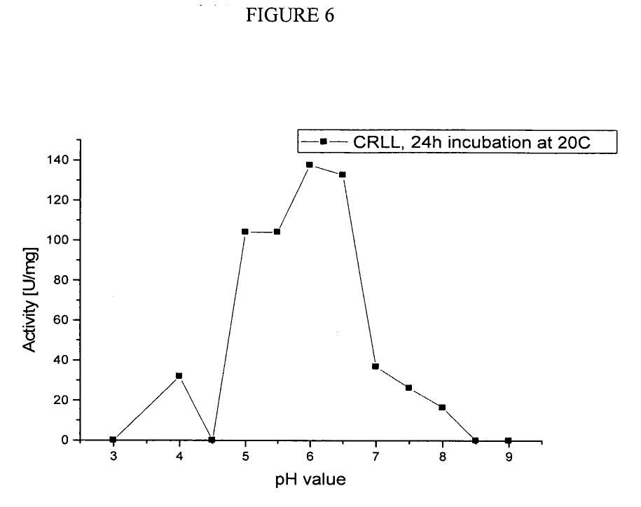

The results demonstrate that LL-CR is active at organic solvent contents up to 50%. Activity of LL-CR in OATS systems compares favorably with the data measured with horse liver ADH (HL-ADH) (FIG. 4): the specific activity of HL-ADH drops off considerably faster than that of LL-CR.

Regeneration of the co-product NADP⁺ can be achieved with isopropanol as the regeneration substrate. Remarkably, the specific activity of LL-CR on isopropanol, between 2-4 U/mg protein, is less dependent on solvent content than that of the oxo compounds and extends beyond 50% solvent content. This demonstrates that neither the LL-CR molecule itself nor the cofactor is unstable in OATS below 60% organic solvent.

The carbonyl reductase from Lactococcus lactis accepts a suitably wide range of hydrophobic ketones at comparable rates to aqueous solvents and is sufficiently stable in monophasic aqueous-organic solvent mixtures. Specific activity is on the same order of magnitude, proving that the cofactor NADP(H) is sufficiently stable in aqueous-organic media of ≦60% acetonitrile, dioxane, or tetrahydrofuran.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

1. An isolated and purified polypeptide comprising:
a microbial polypeptide that has at least 95% sequence identity to a polypeptide having an amino acid sequence of SEQ ID NO:2, wherein the polypeptide has R carbonyl reductase activity.

2. The polypeptide of claim 1, wherein the polypeptide reduces aliphatic ketones, aromatic ketones, alpha ketoesters, beta ketoesters, or a combination thereof.

3. The polypeptide of claim 2, wherein the polypeptide does not reduce keto acids.

4. The polypeptide of claim 1, wherein the polypeptide produces compounds comprising an (R)-specific alcohol functional group.

5. The polypeptide of claim 1, wherein the polypeptide is stable in a solution comprising an organic solvent.

6. The polypeptide of claim 1, wherein the polypeptide is active in a solution comprising about 60% or less of an organic solvent.

7. The polypeptide of claim 1, wherein the polypeptide requires NADP(H) for activity.

8. The polypeptide of claim 1, wherein the polypeptide has a pH optimum of 6.5.

9. A tetramer comprising subunits comprising the polypeptide of claim 1.

10. The polypeptide of claim 1, wherein the polypeptide has a calculated molecular weight of about 28.8 kDa.

11. The polypeptide of claim 1, wherein the polypeptide has a K_{M} of about 0.5 for acetophenone.

12. The polypeptide of claim 1, wherein the polypeptide has a K_{M} of about 0.03 for NADP(H).

13. The polypeptide of claim 1, wherein the polypeptide has a calculated K_{cat} of about 24 s⁻¹.

14. An isolated polypeptide from Lactococcus lactis, wherein the polypeptide is an (R)-specific carbonyl reductase or a fragment thereof and reduces acetophenone or alph,beta-unsaturated carbonyl compound to form a reduced R enantiomeric product.

15. The polypeptide of claim 14, wherein the polypeptide produces a compound comprising an (R)-specific alcohol functional group.

16. The polypeptide of claim 14, wherein the polypeptide comprises at least 80% sequence identity to SEQ ID NO:2.

17. A method for reducing an acetophenone or alph,beta-unsaturated carbonyl compound, comprising:
reacting the acetophenone or alpha, beta-unsaturated carbonyl compound with an isolated polypeptide comprising a sequence having at least about 80% sequence identity to SEQ ID NO:2 or fragment thereof in combination with NADP(H) to form a reduced R enantiomeric product.

18. The method of claim 17, wherein an (R)-specific alcohol functional group is produced.

19. The method of claim 17, wherein the polypeptide reduces the acetophenone or alpha,beta-unsaturated carbonyl compound in the presence of about 10% to about 60% of organic solvent.

20. (canceled)

21. A fusion protein or a chimeric protein comprising the polypeptide of claim 1 or a fragment thereof.

22.-31. (canceled)
